(19) United States
(12) Patent Application Publication
(10) Pub. No.: US 2009/0221950 A1
(43) Pub. Date: Sep. 3, 2009

(54) CATHETER AND TUNNELING DEVICE THEREFOR

(75) Inventor: Joseph R. Atkins, Vandalia, OH (US)

Correspondence Address: Altera Law Group, LLC, 220 S 6 St Suite 1700, Minneapolis, MN, 55402, US

(73) Assignee: GALT MEDICAL CORP., Garland, TX (US)

(21) Appl. No.: 12/434,816

(22) Filed: May 4, 2009

Related U.S. Application Data
(63) Continuation of application No. 11/161,212, filed on Jul. 27, 2005.

(51) Int. Cl. A61M 1/14; A61M 25/06

(52) U.S. Cl. 604/6.16; 606/108

(57) ABSTRACT

A catheter for introduction and removal of fluids from a body and tunneling device therefor, the catheter has a body having a first conduit for removing fluid from the body and a second conduit for delivering fluid to the body wherein at least a part of each of the conduits is integrally formed within a first end of the body and extends along side one another. The first conduit extends beyond the second conduit and forms a terminal point of the first end and defines a first opening thereat and the second conduit extends to a point short of the terminal point defining a second opening. A bumper portion is adjacent the second opening extending toward the terminal point. Another part of each the first and the second conduits are disjoined and form part of a second Y-shaped end. The tunneling device has a channel to removably receive the catheter.

### FIELD OF INVENTION

The present invention relates to catheters and more particularly to an improved catheter for introduction and removal of fluids from a body and a tunneling device to more readily enable insertion of the catheter.

### BACKGROUND OF THE INVENTION

Medical catheters have been used successfully for many years to access the blood stream of human and animal patients. Multi-lumen catheters are used for a variety of applications where it is necessary to have two or more separate fluid pathways. The purposes of accessing the blood circulatory system include introduction of medication, hemodialysis therapy, plasmaphoresis therapy, and other therapies which may require access to the blood circulatory system.

In the case of hemodialysis, a dual-lumen catheter can be employed to simultaneously accommodate opposing blood flow. One lumen aspirates blood from a blood vessel of a patient to a dialysis machine where it is processed for the removal of toxins, while the other lumen infuses the purified blood to the patient.

A problem in many dialysis catheters is that related to clotting and fibrin sheath formation around the “arterial” inlet used for aspiration of blood from a patient. Thrombus and fibrin can occlude distal tips or other places throughout the length of the catheter lumens, resulting in loss of catheter function when such an occlusion prevents blood flow. Usually, catheters have one large exposed hole or one large hole with many little side holes. The thought process is that the extra holes will prevent the inlet from sucking up against the blood vessel wall. Unfortunately, the small holes become filled with fibrin and the large inlet hole develops a trap door clot, which makes the catheter useless. A secondary problem is that related to the arterial lumen “sucking” against the vessel wall in which it resides. This problem can occur if the arterial lumen ports become fully occluded by the patient's vasculature. A technique to resolve such problem is to reverse the bloodlines (i.e., to aspirate through the longer lumen, and to infuse through the shorter lumen, contrary to normal therapeutic pump blood flow). This causes cleaned blood to flow directly toward the lumen that is under vacuum, line reversal in certain catheters can result in inefficient flow and (high re-circulation).

Additionally, there are major side effects which carry patient safety issues, such as hemorrhaging due to the use of indwelling anticoagulants. These anticoagulants are used when the catheter is not being utilized. Side effects of these anticoagulants, primarily heparin, cause acute and chronic gastrointestinal bleeding, retinopathy, and anemia.

Another major issue is that of infection. Currently, catheters on the market use only one single cuff immediately beneath the skin, which is expected to anchor the catheter into the patient's tissue, as well as provide blockage to motile bacteria. This cuff design is often inadequate to perform these functions.

The present invention solves many of the above problems associated with catheters as will become apparent in reading the following hereinafter.

### BRIEF SUMMARY OF THE INVENTION

An object of the invention is to ensure safe access to the blood circulatory system of humans and animals.

Another object of the invention is to enable catheter access to the blood circulatory system of humans and animals without the need for an indwelling anticoagulant.

Yet another object of the invention is to provide a catheter inlet which is substantially less prone to be blocked off as a result of sucking up against the blood vessel wall.

A further object of the invention is to provide a catheter that has the ability to substantially prevent clotting of the lumens of the catheter without the need of clot busting agents.

Another object of the invention is to provide a catheter with the ability to reduce long term infections that accompany long term placement of central venous lines.

A further object of the invention is to improve long term health benefits for hemodialysis and plasmaphoresus patients with improved blood flow rates.

Yet another object of the invention is to provide a catheter that can safely access the blood circulatory system of humans and animals with minimal risk of air embolism.

Accordingly, the present invention is directed to a catheter for introduction and removal of fluids from a body and a tunneling device to more readily enable insertion thereof. The catheter includes a first conduit for removing fluid from the body and a second conduit for delivering fluid to the body wherein at least a part of each of the conduits is integrally connected within a first end of a catheter body and extends along side one another. The connected part of the first conduit, preferably the fluid delivering conduit, extends beyond the second conduit and forms a terminal point of the first end of the catheter and defines a first opening thereat. The connected part of the second conduit, preferably the fluid receiving conduit, extends to a point short of the terminal point and defines a second opening. Also, a bumper portion, which can preferably be tapered, is integrally formed onto the first end adjacent the second opening and extends toward the terminal point.

The first and second conduits each have another part which is disjoined at a second Y-shaped end of the catheter. Each disjoined part of each includes a terminal end defining and opening. Each end can preferably be fitted with a lure lock fitting.

A first flexible rod is provided and is of a size and length to extend through the first conduit in a slidably sealable manner and is equipped with an end cap which is configured to sealably connect to the lure lock fitting of the first conduit. A second flexible rod is provided and is of a size and length to extend through the second conduit in a slidably sealable manner and is equipped with an end cap which is configured to sealably connect to the lure lock fitting of the second conduit. A clamp is operably disposed about each of the conduits.

The first end can include a plurality of longitudinally spaced cuffs which provide for tissue ingrowth therein to enable the catheter to be suitably anchored within the body. A visible indicia can preferably be formed on the first end between the cuffs and the second end to serve as a position indicator of the cuffs.

In addition, the present invention provides for a tunneling device for use in inserting the catheter. The tunneling device includes a relatively rigid member having a handle and an end which is of a length longer than the first end of catheter and is configured with an exposed elongated channel to receive the first end of the catheter. The tunneling device end is equipped with a hollow cap to receive the openings of the first end of the catheter in a manner to maintain the openings substantially covered during insertion of the catheter into the body thereby preventing potential clogging thereof by tissue and blood. The channel can be generally oval shaped to contain the connected conduit parts of the catheter. Further, the tunneling device can include a mid section having a Y-shaped exposed channel continuous with the exposed elongated channel to receive at least part of the Y-shaped end of the catheter.

A method of introducing the catheter into a body is also provided. Further objects and advantages of the present invention will become apparent from the ensuing description and drawings.

### BRIEF DESCRIPTION OF THE DRAWINGS

FIG. 1 shows a catheter of the present invention;

FIG. 2 shows an end portion of the catheter of FIG. 1;

FIG. 3 shows a flexible rod for use in the catheter of FIG. 1;

FIG. 4 shows a tunneling device of the invention;

FIG. 5 shows the tunneling device in FIG. 4 with the catheter of FIG. 1 disposed therein;

FIG. 6 shows a cross-section along line 6-6 of FIG. 5;

FIG. 7 shows the catheter of FIG. 1 with flexible rods therein;

FIG. 8 depicts one step of use of the invention; and

FIG. 9 depicts another step of use on the invention.

### DETAILED DESCRIPTION OF A PREFERRED EMBODIMENT

Referring now to the drawings, the present invention is generally directed to a catheter 10 for introduction and removal of fluids from a body B and a tunneling device 12 to more readily enable insertion of the catheter 10. The catheter 10 includes a first conduit 14 for removing fluid from the body B and another conduit 16 for delivering fluid to the body. Parts 18 and 20 of each of the conduits 14 and 16, respectively, are integrally connected as part a first end 22 of the catheter 10 and. extend along side one another.

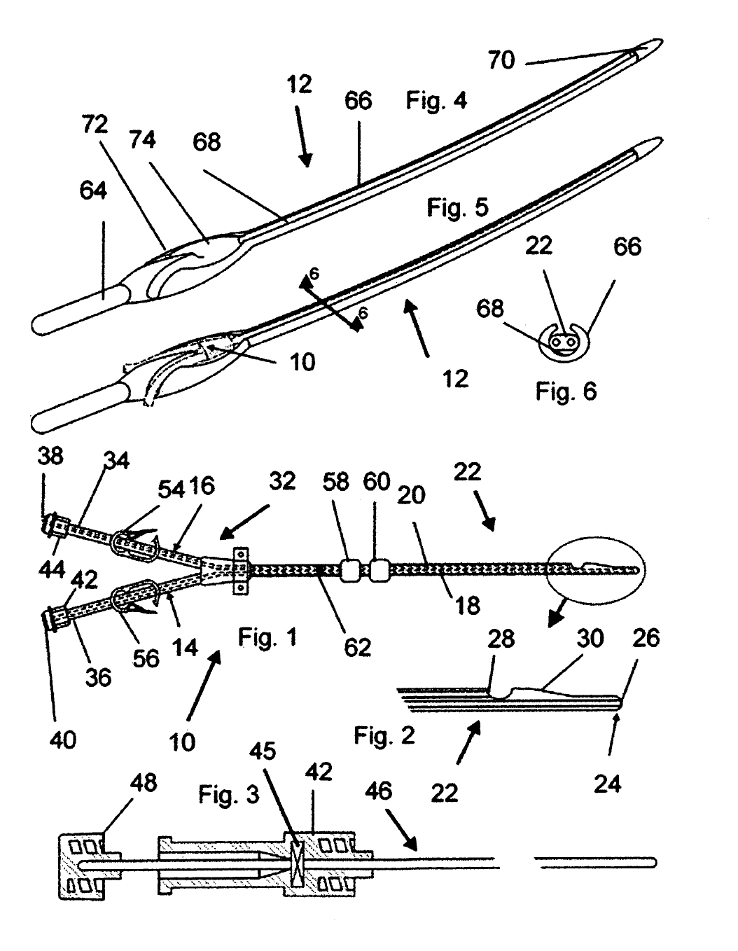

The first conduit 14 can preferably be the fluid delivering conduit (venous flow) which extends beyond the second conduit 16 as best seen in FIGS. 1 and 2 and forms a terminal point 24 of the first end 22 and defines a first opening 26 thereat. The second conduit 16 can preferably be the fluid receiving conduit (arterial flow) which extends to a point short of the terminal point 24 and defines a second opening 28. Also, a bumper portion 30, which can preferably be tapered, is integrally connected to the first end 22 adjacent the second opening 28 and extends toward the terminal point 24.

The first and second conduits 14 and 16 are disjoined at a second Y-shaped end 32 of the catheter 10. Each conduit 14 and 16 includes a terminal end 34 and 36, respectively, defines a respective opening 38 and 40. Each end 34 and 36 extend a sufficient length from the point of connection of parts 18 and 20 to permit easy handling of each during operative procedures by a surgeon. Each end 34 and 36 can preferably be fitted with a lure lock fitting 42 and 44, respectively, which can be separate or part of a flexible rod 46 or 50, and include a respective flexible leaf valve 45 and 51 (not shown) through which the rods 46 or 50 pass through upon insertion/removal of the catheter 10.

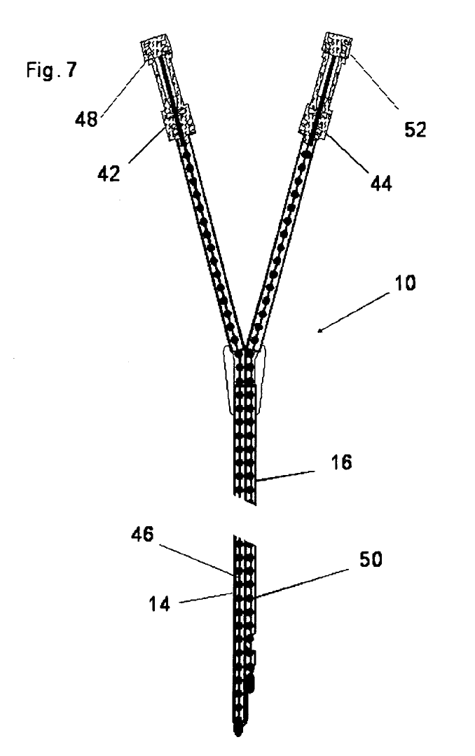

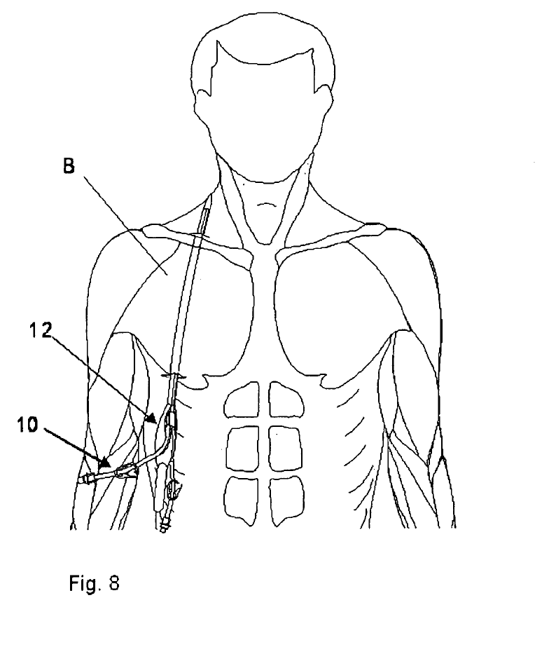

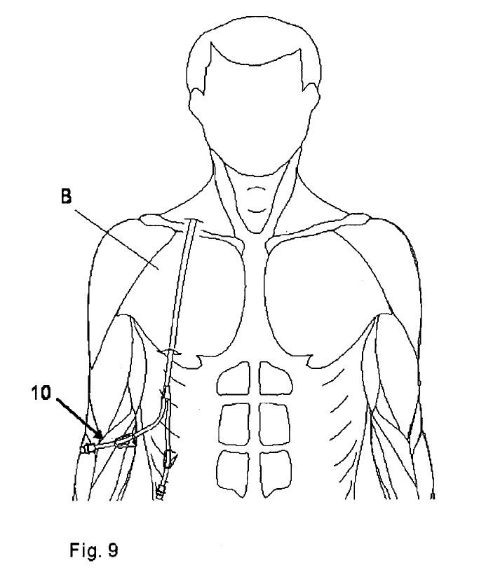

As seen in FIG. 7, the first flexible rod 46 is of a size and length to extend through the first conduit 14 in a slidably sealable manner and is equipped with an end cap 48 which is configured to sealably connect to the lure lock fitting 42 of the first conduit 14. The second flexible rod 50 is of a size and length to extend through the second conduit 16 in a slidably sealable manner and is equipped with an end cap 52 which is configured to sealably connect to the lure lock fitting 44 of the second conduit 16. As can bee seen in FIG. 7, the rods 46 and 50 can include a bead like outer surface which sealably connects to the inner wall of the conduits 14 and 16 to prevent air passage thereby. Clamps 54 and 56 are operably disposed about the conduits 14 and 16, respectively, as seen in FIG. 1.

The first end 22 can include a plurality of longitudinally spaced cuffs 58 and 60 which provide for tissue ingrowth therein to enable the catheter 10 to be suitably anchored within the body B. A visible indicia 62 can preferably be formed on the first end 22 between the cuffs 58 and 60 and the second end 32 to serve as a position indicator of the cuffs 58 and 60 to the surgeon during the insertion procedure.

In addition, the present invention provides for the tunneling device 12 for use in inserting the catheter 10. The tunneling device 12 can be a single piece construction. The tunneling device 12 can be of a relatively rigid material, such as medical grade stainless steel or synthetic plastic, e.g., polyurethane. The tunneling device 12 includes a handle 64 and an end 66 which is of a length longer than the first end 22 of catheter 10 and is configured with an exposed elongated channel 68 to receive the first end 22 of the catheter 10. The tunneling device end 66 is equipped with a hollow cap 70 to the to receive the openings 26 and 28 of the first end 22 of the catheter 10 in a manner to maintain the openings 26 and 28 substantially covered during insertion of the catheter 10 into the body B thereby preventing potential clogging thereof by tissue and blood. The cap 70 can be a bullet like tip which can be threadably connected to the end 66. The channel 68 can be generally oval shaped in cross section as seen in FIG. 6 to accept the end 22. Further, the tunneling device 12 can include a mid section 72 having a Y-shaped exposed channel 74 continuous with the exposed elongated channel 68 to receive at least part of the Y-shaped end 32 of the catheter 10.

A method of introducing the catheter into a body is also provided. The catheter 10 and tunneling device 12 can come pre-packaged for a surgeon with the catheter 10 already loaded into the tunneling device 12. Once the surgeon has pushed the tunneling device 12 through the tissue of the body B to the incision at the internal jugular, the cap 70 is removed. The surgeon grasps the end 22 of the catheter 10 at the incision, lifts the Y-shaped end 32 of the catheter 10 from the Y-shaped exposed channel 74 and slowly removes the tunneling device 12. The exposed elongated channel 68 of the tunneling device 12 allows it to slip around the catheter 10 it is removed. The tunneling device 12 is only intended for a single insertion. After it has accomplished its task, it is dispensed. The invention uses flexible rods 46 and 50 to displace the blood in the catheter conduits 14 and 16, when the catheter 10 is not being used.

With the present invention, there is a substantially reduced or elimination of risk of hemorrhaging due to the use of indwelling anticoagulants, acute and chronic gastrointestinal bleeding, retinopathy, and anemia. Further, the dual cuffs 58 and 60 provide for improved anchoring into the patient's tissue as well as provide blockage to motile bacteria wherein the indicia 62 indicates to the surgeon when the cuffs 58 and 60 are close to exiting the incision site.

The above described embodiment is set forth by way of example and is not for the purpose of limiting the present invention. It will be readily apparent to those skilled in the art that obvious modifications, derivations and variations can be made to the embodiment without departing from the scope of the invention. Accordingly, the claims appended hereto should be read in their full scope including any such modifications, derivations and variations.

What is claimed is:

1. A catheter and for introduction and removal of fluids from a body, which includes: a catheter body having a first conduit for removing fluid from the body and a second conduit for delivering fluid to the body wherein a first part of each of the conduits is connected within a first end of said catheter body and extend along side one another, said connected first part of said first conduit extends beyond said connected first part of said second conduit and forms a terminal point of said first end and defines a first opening thereat and said second conduit extends to a point short of said terminal point defining a second opening, a bumper portion connected to said first end adjacent said second opening and extends toward said terminal point, and wherein a second part of each said first conduit and said second conduit are disjoined and form part of a second Y-shaped end of said catheter and said second part of each said conduit includes a terminal end defining and opening.

2. The catheter of claim 1, wherein said bumper is tapered.

3. The catheter of claim 1, wherein said bumper is integrally connected to said first end.

4. The catheter of claim 1, wherein each said disjoined conduit part extend a length to readily enable handling thereof during operation.

5. The catheter of claim 1, wherein each terminal end of said disjoined parts include a lure lock fitting.

6. The catheter of claim 5, which includes a first flexible rod of a size and length to extend through said first conduit in a slidably sealable manner and is equipped with an end cap which is configured to sealably connect to said lure lock fitting of said first conduit and a second flexible rod is of a size and length to extend through said second conduit in a slidably sealable manner and is equipped with an end cap which is configured to sealably connect to said lure lock fitting of said second conduit.

7. The catheter of claim 1, which further includes a clamp operably disposed about each of said disjoined conduit parts.

8. The catheter of claim 1, which further includes a plurality of longitudinally spaced cuffs disposed on said first end of said catheter which provide for tissue ingrowth therein to enable said catheter to be suitably anchored within the body.

9. The catheter of claim 1, which further includes a cuff disposed on said first end of said catheter which provides for tissue ingrowth therein to enable said catheter to be suitably anchored within the body and a visible indicia formed on said first end between said cuff and said second end of said catheter to serve as a position indicator of said cuff.

10. A tunneling device for use in inserting a catheter, which includes: a relatively rigid member having a handle and an end configured with an exposed elongated channel to receive the catheter, wherein said end is equipped with a hollow cap to receive openings of a first end of the catheter in a manner to maintain the openings substantially covered during insertion of the catheter into the body thereby preventing potential clogging thereof by tissue and blood.

11. The tunneling device of claim 10, wherein said elongated channel is generally oval shaped in cross section.

12. The tunneling device of claim 10, which further includes a mid section having a Y-shaped exposed channel continuous with said exposed elongated channel to receive at least part of a Y-shaped end of the catheter.

13. A method of introducing the catheter into a body, which includes the steps of:
(a) providing a tunneling device for use in inserting a catheter, wherein said tunneling device includes a relatively rigid member having a handle and an end configured with an exposed elongated channel to receive the catheter, wherein said end is equipped with a removable hollow cap to receive openings of a first end of the catheter in a manner to maintain the openings substantially covered during insertion of the catheter into the body thereby preventing potential clogging thereof by tissue and blood;
(b) laying an end of the catheter through the exposed elongated channel in a manner such that end openings of the catheter are received in the cap of the tunneling device; and
(c) inserting the tunneling device having the catheter therein through a first incision of a body such that the cap extends out a second incision of the body;
(d) removing the cap; and
(e) lifting the first end of catheter from the tunneling device to enable removal of the catheter from the tunneling device and removal of the tunneling device from the body.
